(19) United States
(12) Patent Application Publication
(10) Pub. No.: US 2025/0087333 A1
(43) Pub. Date: Mar. 13, 2025

(54) SYSTEM AND METHOD FOR IMPROVING A COGNITIVE STATE OF A PATIENT THROUGH CUSTOMIZED MUSIC THERAPY

(71) Applicant: Cheryl Meier-Keller, ALISO VIEJO, CA (US)

(72) Inventor: Cheryl Meier-Keller, ALISO VIEJO, CA (US)

(21) Appl. No.: 18/770,649

(22) Filed: Jul. 11, 2024

Related U.S. Application Data
(60) Provisional application No. 63/526,186, filed on Jul. 12, 2023.

(51) Int. Cl. G16H 20/70

(52) U.S. Cl. CPC G16H 20/70

(57) ABSTRACT

A method, process, and system to treat a psychological issue of a user by providing customized therapy media to the user based on the psychological state of the user. The method comprising a) defining a problem of a user, b) determining a target outcome the user needs to overcome the problem, c) selecting at least one thought the user needs to overcome the problem, d) having the user concentrate on the at least one thought the user needs to overcome the problem and achieve the target outcome of the user, e) searching at least one media that corresponds to the thinking needed to achieve the target outcome of the user. The methods, processes, and systems can leverage the capabilities of artificial intelligence algorithms, non-transitory storage medium, and a processor to identify user problems, determine target outcomes, select relevant thoughts, search for appropriate media, and anchor users to the selected media.

### CROSS REFERENCE TO RELATED APPLICATIONS

The present application is a United States Non-Provisional Patent Application. This Non-Provisional patent application claims priority to the co-pending United States patent provisional application having the Ser. No. 63/526,186, filed Jul. 12, 2023 with confirmation number 7355. United States patent provisional application having the Ser. No. 63/526,186 is incorporated in its entirety by reference.

### FIELD OF INVENTION

The present invention relates to treating psychological issues in humans. The present invention further relates to methods, processes, and systems for delivering therapy to users and, more specifically, to a system that provides customized therapy media based on the user's psychological state. More specifically, the present invention relates to treating patients with psychological issues using music therapy. Even more particularly, the present invention provides a system and a method to provide customized songs based on the psychological condition of the user. In a more specific application, the system leverages the capabilities of artificial intelligence algorithms, non-transitory storage medium, and a processor to identify user problems, determine target outcomes, select relevant thoughts, search for appropriate media, and anchor users to the selected media.

### BACKGROUND OF THE INVENTION

The psychology of a person is acknowledged as a valuable indicator for various diseases such as dementia, depression, anxiety, and Attention Deficit Hyperactivity Disorder. Monitoring the psychological state of patients has therefore become important in assessing the overall health of patients as mental conditions can often lead to or affect the physical condition of the patients after some time.

Somatization, for example, is an expression used to explain the manifestation of psychological distress by the presentation of physical symptoms. Somatic symptom disorder and related disorders are characterized by persistent physical symptoms that are associated with excessive or maladaptive thoughts, feelings, and behaviors in response to these symptoms and associated health concerns. Throwing up from anxiety, having a headache due to stress, or feeling physically weak after trauma are all examples of somatization. But these instances are typically situational and temporary. Somatization becomes a clinical issue when it causes prolonged and severe distress. Disorders characterized by somatization extend in a continuum from those in which symptoms develop unconsciously and non-volitionally to those in which symptoms develop consciously and volitionally. Somatic symptoms may not have an observable cause, but the pain and distress are real. An individual may interpret their symptoms as a bodily illness and see a physician. But the doctor will rarely find a physical explanation for the person's symptoms. Even if the doctors find a physical problem, the symptoms will likely be unrelated to or out of proportion with the person's condition.

Recent research into psychological disorders has shown that most patients have experienced traumas bodily either in their childhood or at least some point in their life. For example, someone who broke their ankle in a sports game may report breathing issues. In all of the disorders, patients focus prominently on somatic concerns. These disorders are distressing and often impair social, occupational, academic, or other aspects of functioning. Thus, somatization typically leads patients to seek medical evaluation and treatment rather than psychiatric care.

The conventional approach to psychological therapy faces a challenge where patients have to schedule clinic appointments; visit hospitals within those reserved times and follow a predetermined therapeutic program. This limitation prevents the customization of therapy to individual characteristics. However, by incorporating ubiquitous technology into the field of psychological therapy, individuals can receive continuous and tailored therapeutic services anytime and anywhere, enhancing the effectiveness of psychological treatment.

Current therapies now include “resomatization” in the healing process. This means that the most effective therapies are getting our bodies involved in healing. For example, eye movement desensitization and reprocessing (EMDR) involves moving your eyes a specific way while you process traumatic memories which was initially used for the treatment of post-traumatic stress disorder. This is why people have gravitated towards yoga or EMDR, or equestrian therapies and found those highly successful. However, not everyone has access to horses, nor will they require of themselves the discipline needed to do a regular practice of yoga, because they do not realize the lasting benefits to their mental health these kinds of practice could bring to them.

Music therapy means using music to restore, maintain and improve mental and physical health for therapeutic purposes. Music is seen as a complex set of different processes and characteristics such as pitch, pitch interval, tempo, rhythm, melodic contour, and rhythmic contour. Scientific evidence has been produced about various clinical conditions concerning musically trained and untrained individuals. This field of research has emerged as a specialized branch of cognitive neuroscience and psychology and is variously referred to as neuro-musicology, music cognition, or music psychology. Through music therapy, unnecessary worries and anxiety can be avoided, and adaptation to society is improved, so that the recurrence can be prevented. Music, therefore, is an apt intervention to address multiple domains, such as cognitive, emotional, physical, and social domains of functioning, which are adversely affected in psychiatric conditions.

Traditional psychological therapy often relies on standardized approaches that may not adequately address the unique needs of individual users. Customized therapy tailored to the specific psychological state of the user can significantly enhance treatment outcomes. Furthermore, providing therapy media, such as songs with lyrics or videos, that correspond to the user's thoughts and desired outcomes can help facilitate the achievement of those outcomes.

The present invention aims to address the challenges encountered in conventional psychological therapy services. Its objective is to offer a psychological health management system and method that can deliver real-time music therapy to users based on their condition and psychological state, free from limitations of time and location. Accordingly, embodiments of this invention satisfy these needs.

Another aim of embodiments of the present invention is to offer a psychological health management system and method that can provide real-time music therapy tailored to the user's changing thoughts and psychological condition. This is achieved by adapting the psychological therapy according to the transition from the user's previous condition to their current thoughts and psychological condition to achieve the desired outcome. As the users' thought pattern changes or improves new and different songs and or videos may be utilized to get the user to the desired thoughts and psychological state.

Another object of embodiments of the present invention is to provide a psychological health management system and method which can primarily determine a cognitive pattern or thought process associated with the user's condition and determine an alternative thought process or pattern along with a song matching said alternative thought process which is required to achieve a final therapeutic outcome for the user.

1. Another object of embodiments of the present invention is to provide a platform where Artificial Intelligence (AI) and Machine Learning (ML) can assist the user and/or treating professionals in treating psychological issues.

### SUMMARY OF THE INVENTION

In one embodiment, a method to treat a psychological issue of a user by providing customized therapy media to the user based on the psychological state of the user is disclosed. The method comprises a) defining a problem of a user; b) determining a target outcome, the user needs to overcome the problem; c) selecting at least one thought the user needs to overcome the problem; d) having the user concentrate on the at least one thought the user needs to overcome the problem and achieve the target outcome of the user; e) searching at least one media that corresponds to the thinking needed to achieve the target outcome of the user, wherein the media is selected from a song with lyrics and/or a video; f) having the user anchor to the at least one media with the user achieving the target outcome; and g) treating the user by having the user playing the media multiple times to achieve the targeted outcome.

In a second embodiment, a computer-implemented method of providing customized therapy media to a user based on the psychological state of the user is disclosed. The method comprises a) identifying, via a user device connected to a server over a network, a psychological problem of the user; b) determining, from a database connected to the server, a target outcome for the user to overcome the problem as identified; c) selecting, by the server, at least one thought corresponding to the target outcome the user needs to overcome the problem; d) searching, in a media database coupled to the network and having a plurality of media, one or more media that correspond to the problem and the target outcome the user needs using artificial intelligence; and e) anchoring the user, via the user device, to at least one media from said one or more media to achieve the target outcome.

In a third embodiment, a system for providing customized therapy media to a user based on the psychological state of the user. The system comprises a non-transitory storage medium; and a processor coupled to the non-transitory storage medium, configured at least to: identify, via the user device, a psychological problem of the user; determine, from a database connected to the server, a target outcome for the user to overcome the problem as identified; select, by the server, at least one thought corresponding to the target outcome the user needs to overcome the problem; search, in a media database coupled to the network and having a plurality of media, one or more media that correspond to the problem and the target outcome the user needs using artificial intelligence; and anchor the user, via the didactic teaching of the trained therapist and the user device, to at least one media from said one or more media to achieve the target outcome.

In a fourth embodiment, a method to treat a psychological issue of a user is disclosed. the method comprises a) defining a problem of a user; b) determining a target outcome, the user needs to overcome the problem; c) selecting at least one thought the user needs to overcome the problem; d) having the user concentrate on the at least one thought the user needs to overcome the problem and achieve the target outcome of the user; e) searching at least one media that corresponds to the thinking needed to achieve the target outcome of the user, wherein the media is selected from a song with lyrics and a didactic psychological teaching video; f) providing customized therapy media to the user based on the psychological state of the user; g) having the professional work with the user to anchor to the at least one media with the user achieving the target outcome; and h) treating the user by having the user playing the media and/or the teaching video multiple times to achieve the targeted outcome.

In a fifth embodiment, a computer-implemented method is disclosed. The method comprises a) identifying, via the user device connected to a server over a network, a psychological problem of the user; b) determining, from a database connected to the server, a target outcome for the user to overcome the problem as identified; c) selecting, by the server, at least one thought corresponding to the target outcome the user needs to overcome the problem; d) searching, in a media database coupled to the network and having a plurality of media, one or more media that correspond to the problem and the target outcome the user needs using artificial intelligence; e) providing a customized therapy media to the user based on psychological state of the user; and f) anchoring the user, via the trained professional and the user device, to at least one media from said one or more media to achieve the target outcome.

In a sixth embodiment, a system is disclosed. The system comprises a non-transitory storage medium; and a processor coupled to the non-transitory storage medium, configured at least to: identify, via a user device, a psychological problem of the user; determine, from a database connected to the server, a target outcome for the user to overcome the problem as identified; select, by the server, at least one thought corresponding to the target outcome the user needs to overcome the problem; search, in a media database coupled to the network and having a plurality of media, one or more media that correspond to the problem and the target outcome the user needs using artificial intelligence for providing a customized therapy media to the user based on psychological state of the user; and anchor the user, via the didactic teaching of the trained therapist and the user device, to at least one media from said one or more media to achieve the target outcome.

### BRIEF DESCRIPTION OF DRAWINGS

The above and other objects, features, and advantages of the present invention will be apparent from the following detailed description of the preferred embodiments of the invention in conjunction with the accompanying drawings, in which:

FIG. 1 illustrates a flowchart highlighting therapy steps, in accordance with an embodiment of the present invention;

FIG. 2 illustrates a flow chart illustrating an implementation of a psychological health management system, in accordance with an embodiment of the present invention; and

FIG. 3 illustrates an exemplary network architecture for implementing a psychological health management system, in accordance with an embodiment of the present invention;

FIG. 4 illustrates the psychological health management system of FIG. 3.

Additional objects, advantages, and novel features of the invention will be set forth in part in the description which follows and will become apparent to those skilled in the art upon examination of the following or may be learned by practice of the invention. The objects and advantages of the invention may be realized and attained by means of the instrumentalities and combinations particularly pointed out in the appended claims.

### DETAILED DESCRIPTION

The following description of the embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. If the specification states a component or feature “may”, “can”, “could”, or “might” be included or have a characteristic, that particular component or feature is not required to be included or have the characteristic.

The inclusion of value ranges in this document is intended solely as a convenient way to refer to each specific value within the range individually. Unless explicitly stated otherwise, each individual value is considered part of the specification as if it had been separately mentioned herein. Furthermore, all methods described in this document can be carried out in any appropriate sequence, unless otherwise specified or clearly contradicted by the context. The utilization of any examples or exemplary language (e.g., “such as”) in relation to specific embodiments presented herein is solely intended to enhance the understanding of the invention and should not be interpreted as imposing any limitations on the scope of the invention as otherwise claimed. None of the language used in the specification should be understood as indicating any element essential to the practice of the invention that is not explicitly claimed.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

The terms “patient” and “user” mentioned in the following description are intended to encompass a wide range of individuals, including patients, students, prisoners, residents, therapy seekers, and others associated with an institution. In a general sense, a user, resident, patient, student, therapy seeker, and similar designations are individuals who receive therapy services. Similarly, the terms “doctor” and “therapist”, which will be used frequently within the context of the following description, are to be broadly construed as referring to any of a doctor, medical practitioner, psychologist, psychiatrist, therapist, and the like, who are involved in the process of delivering therapy to patient or therapy seeker.

Within the context of the following description, the terms “music,” “content,” “media,” and similar expressions will be utilized extensively to describe particular therapies provided to a patient. It is important to interpret these terms in a broad manner, as they are largely interchangeable when referring to the therapy being delivered unless explicitly defined otherwise.

The term “psychological condition or state” or “measure of psychological ability or function,” as used herein, may refer to a representation of the state of the user's mental processes of perception, memory, judgment, reasoning, and/or the like. In some embodiments, the representation can be for a specific type of function (e.g. memory). In some embodiments, the representation can be for several types of functions (e.g., memory and perception). In some embodiments, the representation can pertain to all of them as a whole.

The terms “Artificial Intelligence” (AI) as used in the present application is a broad field focused on creating intelligent systems that can perform tasks that typically require human intelligence. In general, AI systems aim to simulate human cognitive abilities such as learning, reasoning, problem-solving, and decision-making. They accomplish this by employing a combination of algorithms, data, and models. At its core, AI involves the development of software systems that can process and analyze vast amounts of data, extract meaningful patterns, and make predictions or take actions based on the acquired knowledge.

The term “Machine Learning” (ML) as used in the present application is a subfield of AI that enables computers to learn and improve from experience without being explicitly programmed. ML algorithms are designed to automatically analyze and interpret data, identify patterns, and make predictions or decisions based on the identified patterns. The process typically involves three key components: data, models, and optimization. First, large volumes of relevant data are collected and preprocessed to make it suitable for analysis. Then, ML models, such as neural networks or decision trees, are trained using this data to learn patterns and relationships. Finally, optimization techniques are employed to fine-tune the models, making them more accurate and efficient in their predictions or actions.

The present invention relates to a system and method to treat a psychological issue of a user by providing customized therapy media to the user based on the psychological state of the user. The system and method as disclosed herein below can be employed in conjunction with a medical establishment or medical organization, such as a hospital, physician's clinic, pain management center, psychotherapist's practice, and similar settings. The objective of the present invention is to collect mental health information for a patient using validated psychological evaluations. By analyzing the outcomes of these assessments, the method or treating professional can automatically determine the psychological conditions that are affecting the patient the most. Additionally, the method, through AI and ML can offer automated suggestions and advice on strategies to address and overcome these psychological conditions, either as an independent treatment or in combination with prescribed medications.

To achieve the above objectives and in accordance with one embodiment, the present invention discloses a psychological health management method and a system for providing a customized psychological therapy comprising at least one thought and at least one media based on a psychological state of a user. The method and system of the present invention are based on a seven-step process comprising: a) defining a problem of a patient; b) identifying a target outcome the patient needs to overcome the problem; c) determining at least one thought the patient needs to overcome the problem; d) having the patient concentrate on the at least one thought needed to achieve the target outcome of the patient; e) finding at least one song with lyrics that correspond to the thinking needed to achieve the target outcome of the patient; f) having the patient anchor the at least one song with the patient achieving the target outcome; and g) treating the patient by having the patient playing the song multiple times to achieve the targeted outcome.

In accordance with an embodiment, the method of treating a psychological issue of a user comprises defining a problem of a user; determining a target outcome that the user needs to overcome the problem; selecting at least one thought that the user needs to overcome the problem; having the user concentrate on the at least one thought that the user needs to overcome the problem and achieve the target outcome of the user; searching at least one media that corresponds to the thinking needed to achieve the target outcome of the user, wherein the media is selected from a song with lyrics and a video; having the user anchor to the at least one media with the user achieving the target outcome; and treating the user by having the user playing the media multiple times to achieve the targeted outcome.

In accordance with a further embodiment, the method further comprises creating a video didactic teaching of the selected at least one media where the at least one media is a song with lyrics, and treating the patient by having the patient play the video multiple times to achieve the targeted outcome.

In another embodiment, the method comprises determining effectiveness of the at least one media in achieving the target outcome and changing the at least one media. In yet another embodiment, the method further comprises determining effectiveness the at least one thought the user needs to overcome the problem and changing the at least one thought the user needs to overcome the problem.

In accordance with another aspect, the present invention discloses a computer-implemented method of providing customized therapy media to a user based on a psychological state of the user, wherein said computer-implemented method comprising: a) identifying, via a user device connected to a server over a network, a psychological problem of the user; b) determining, from a database connected to the server, a target outcome for the user to overcome the problem as identified; c) selecting, by the server, at least one thought corresponding to the target outcome the user needs to overcome the problem; d) searching, in a media database coupled to the network and having a plurality of media, one or more media that correspond to the problem and the target outcome the user needs using artificial intelligence; and e) anchoring the user, via the user device, to at least one media from said one or more media to achieve the target outcome.

In accordance with an aspect of the computer-implemented method, the step of determining at least one thought includes determining at least one alternative thought the user needs to achieve the target outcome preferably with the aid of the trained professional who assesses the “right fit” of the artificial intelligence's selection, provides didactic teaching of important, higher-level psychological adaptations and concepts, utilizing the vessel/frame of the therapy media. The preferred and optimal process model is to include the trained professional in the process even if AI and ML could perform the entire process autonomously. The trained professional will be able to spot and fix any mistakes and mismatches caused by AI and ML.

In yet another aspect of the invention, the step of anchoring the computer-implemented method includes playing the (now “unpacked” or in-depth psychologically explained) at least one media multiple times in a loop till the target outcome is achieved or in case, achieving the target outcome is delayed after a predetermined time, changing the thought the user needs to overcome the problem. In a preferred embodiment of the present invention, one or more media includes a plurality of audio/video material including audio/video songs with lyrics as well as didactic psychological teaching. The didactic psychological teaching is preferably performed live with the client or in a video format. In the video format, the didactic teaching of that song can be reused for others who match that song.

In a further aspect of the invention, the computer-implemented method comprises receiving from the user device, a compliance report on listening to the at least one media by automatically acquiring a daily report. In a preferred embodiment, the computer-implemented method comprises receiving a review response from the user via the user device on the at least one media to improve the step of searching for relevant media through machine learning. In an alternate embodiment, the computer-implemented method further comprises receiving a response from the user while focusing on the at least one thought or an alternative thought; and changing the media based on a change in the at least one thought or the alternative thought.

In some embodiments of the present invention, the step of identifying the problem in said computer-implemented method includes receiving from the user, via the user device, responses to a psychological evaluation test provided to the user on the user device, wherein the user device is adapted to receive user inputs via a graphical user interface.

In accordance with a further embodiment, the step of identifying the target outcome comprises comparing, with a database of known psychological conditions and their respective traits, the identified problem of the user.

In an aspect of the invention, the step of anchoring the user in the computer-implemented method comprises setting aside time to learn from, understand, and begin to apply the didactic teaching provided by the trained professional, and then setting a duration for which the at least one media is to be played to achieve the target outcome.

In a preferred embodiment, the computer-implemented method further comprises monitoring a variation in the psychological state of the user at regular intervals in course to achieve the target outcome.

In an alternate embodiment, the computer-implemented further comprises using artificial intelligence by the server over the network to identify the psychological problem of the user. Furthermore, the computer-implemented comprises using artificial intelligence by the server to determine from the database the target outcome for the user to overcome the problem as identified. The computer-implemented method further comprises using artificial intelligence by the server to select the at least one thought corresponding to the target outcome the user needs to overcome the problem. In a preferred embodiment, the computer-implemented method comprises using artificial intelligence by the server to search and select the at least one media from said one or more media in a media database to achieve the target outcome.

In another aspect of the present invention, a system for providing customized therapy media to a user based on the psychological state of the user is disclosed. Said system includes a non-transitory storage medium; and a processor coupled to the non-transitory storage medium, configured at least to identify, via the user device, a psychological problem of the user; determine, from a database connected to the server, a target outcome for the user to overcome the problem as identified; select, by the server, at least one thought corresponding to the target outcome the user needs to overcome the problem; search, in a media database coupled to the network and having a plurality of media, one or more media that correspond to the problem and the target outcome the user needs using artificial intelligence; and anchor the user, via the user device and the intervention provided by the trained professional, to at least one media from said one or more media to achieve the target outcome.

In an alternate aspect of the present invention, the system further comprises an additional computer system connected to the server over the network to display the psychological problem, the target outcome, the suggested at least one thought, and the selected at least one media to a therapist for further evaluation.

In a preferred embodiment, the non-transitory storage medium is configured to store details of the user including the user's medical history. To protect the user's privacy, an encrypted program, preferably at the very beginning (sign up), can be utilized that assigns the user a number which they can create a username for. Therefore, any data collected can be used to create better outcomes in the future and it is in no way connected to the identity of the actual user.

In another aspect of the invention, the system further comprises an additional database connected to the server over the network and configured to store the user details, including the user's medical history, the psychological problem, the target outcome, the suggested at least one thought, and the at least one media suggested to each of the users.

In another aspect of the invention, the system further comprises allowing the user to input the symptoms or issues in his language, using artificial intelligence to convert the inputted data to psychological terms, using the converted data to search the physiological database to determine the psychological issue or issues, suggesting thought patterns that can help improve the psychological conditions and the displaying the thought patterns to the user and/or treating professional to pick one or more thought patterns and using artificial intelligence to suggest songs that have similar language patterns to the chosen thought pattern to help the user anchor the new thought patterns.

The computer system located remotely may consist of a remote server, a remote computer network, a distributed network, or any other appropriate type of computer system. This remote computer system is configured to gather various data, including the psychological test results; search results obtained from the method; patient demographic data; patient medical records; medical events; and/or feedback regarding patient psychological condition improvements, etc., provided by patients after therapy sessions. Additionally, it can remotely analyze and manipulate psychological test results, search outcomes, and patient feedback accumulated over a period of time. This analysis aids in selecting the subsequent course of action or customizing guided parameters for the next course of action for a patient within a specific patient population.

In some aspects, systems may be implemented on a distributed computing network, such as one having any number of clients and/or servers. According to the aspect, any number of clients may be provided. Each client may run software for implementing client-side portions of a system; clients may comprise a system. In addition, any number of servers may be provided for handling requests received from one or more clients. Clients and servers may communicate with one another via one or more electronic networks, which may be in various aspects any of the Internet, a wide area network, a mobile telephony network (such as CDMA or GSM cellular networks), a wireless network (such as Wi-Fi, WiMAX, LTE, and so forth), or a local area network (or indeed any network topology known in the art; the aspect does not prefer any one network topology over any other).

Networks may be implemented using any known network protocols, including for example wired and/or wireless protocols. In addition, in some embodiments, servers may call external services when needed to obtain additional information or to refer to additional data concerning a particular call. Communications with external services may take place, for example, via one or more networks. In various aspects, external services may comprise web-enabled services or functionality related to or installed on the hardware device itself.

Devices that are in communication with each other need not be in continuous communication with each other unless expressly specified otherwise. In addition, devices that are in communication with each other may communicate directly or indirectly through one or more communication means or intermediaries, logical or physical.

As used herein, the term “software” is meant to be synonymous with any code or program that can be in a processor of a host computer, regardless of whether the implementation is in hardware, firmware, or as a software computer product available on a disc, a memory storage device, or for download from a remote machine. The embodiments described herein include such software to implement the equations, relationships, and algorithms described above. One skilled in the art will appreciate further features and advantages of the illustrated embodiments based on the above-described embodiments. Accordingly, the illustrated embodiments are not to be limited by what has been particularly shown and described, except as indicated by the appended claims. All publications and references cited herein are expressly incorporated herein by reference in their entirety

In one embodiment, the present invention encompasses a system, architectures, methods, mechanisms, and devices capable of effectively resolving numerous shortcomings found in the prior art concerning the generation of media content. Specifically, it focuses on creating an audio and/or video playlist that is designed to elicit a physiological response in a listener, aligning with a desired outcome category. This physiological response can include a heightened mental capacity to understand and hold psychological, spiritual, and philosophical insights and produce a desired outcome.

In accordance with one embodiment, the present invention discloses a method to treat a psychological issue of a user by providing a customized therapy comprising at least one thought and at least one media to the user based on the psychological state of the user. In an embodiment, the method includes the following steps:
defining a problem of a user;
determining a target outcome the user needs to overcome the problem;
selecting at least one thought the user needs to overcome the problem;
having the user concentrate on the at least one thought the user needs to overcome the problem and achieve the target outcome of the user;
searching at least one media that corresponds to the thinking needed to achieve the target outcome of the user, wherein the media is selected from a song with lyrics and a professionally trained didactic teaching video;
having the user or therapist anchor the user to the at least one media with the user achieving the target outcome; and
continue treating the user by having the user play the media multiple times to achieve the targeted outcome.

In accordance with another embodiment, the present invention discloses a computer-implemented method of providing customized therapy media to a user based on the psychological state of the user, wherein said computer-implemented method includes: a) identifying, via a user device connected to a server over a network, a psychological problem of the user;
b) determining, from a database connected to the server, a target outcome for the user to overcome the problem as identified;
c) selecting, by the server, at least one thought corresponding to the target outcome the user needs to overcome the problem;
d) searching, in a media database coupled to the network and having a plurality of media, one or more media that correspond to the problem and the target outcome the user needs using artificial intelligence; and
e) anchoring the user, via the trained therapist's didactic teaching and the user device, to at least one media from said one or more media to achieve the target outcome.

In accordance with another aspect, the present invention is directed to a system for providing customized therapy media to a user based on the psychological state of the user. In an embodiment, the system comprises: a non-transitory storage medium; and a processor coupled to the non-transitory storage medium, configured at least to:
identify, via the user device, a psychological problem of the user; determine, from a database connected to the server, a target outcome for the user to overcome the problem as identified;
select, by the server, at least one thought corresponding to the target outcome the user needs to overcome the problem; search, in a media database coupled to the network and having a plurality of media, one or more media that correspond to the problem and the target outcome the user needs using artificial intelligence; and anchor the user, via the trained therapist's didactic teaching and the user device, to at least one media from said one or more media to achieve the target outcome.

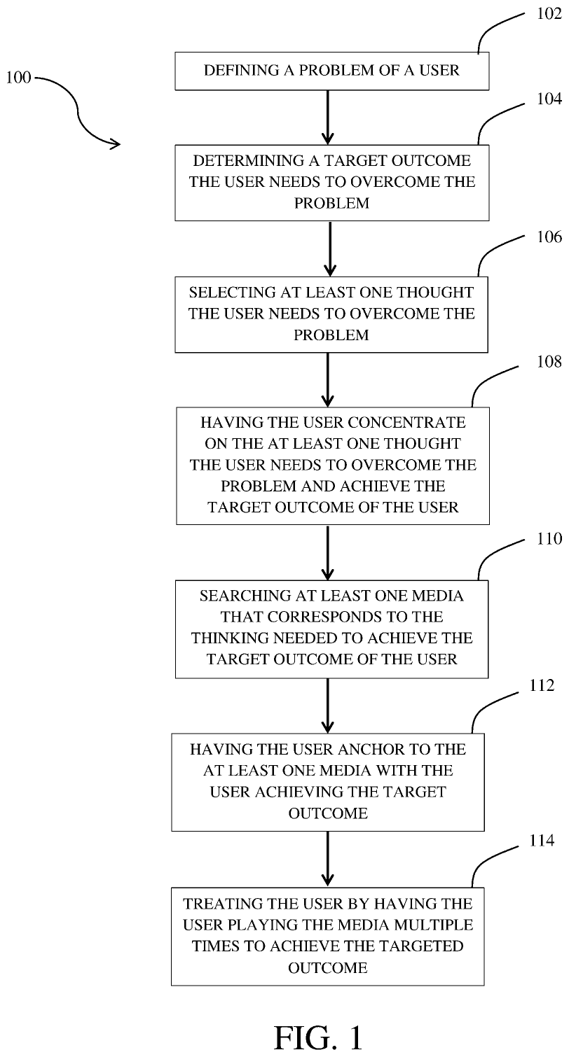

FIG. 1 illustrates a method flowchart 100 highlighting the steps for implementing psychological therapy for a patient. In accordance with an embodiment, the method 100 is a seven-step therapy method for treating a psychological problem of a user. The method 100 involves the following steps: defining a problem of a user or a patient in Block 102; determining a target outcome the user needs to overcome the problem in Block 104; selecting at least one thought the user needs to overcome the problem in Block 106; having the user concentrate on the at least one thought the user needs to overcome the problem and achieve the target outcome of the user in Block 108; searching at least one media that corresponds to the thinking needed to achieve the target outcome of the user in Block 110; having the user anchor to the at least one media with the user achieving the target outcome in Block 112, and treating the user by having the user playing the media multiple times to achieve the targeted outcome in Block 114.

In accordance with an embodiment, the step of defining the psychological problem affecting a user in Block 102 includes determining a state of mind of the user. The psychological problem may include, but is not limited to, anxiety, depression, substance abuse, physical pain, post-traumatic stress disorder (PTSD), and the like. The step of defining may also involve ranking or prioritizing a user's psychological conditions, such as based on the known relationship between various psychological conditions and intensities of these conditions as determined from various psychological tests conducted on a patient. For instance, to determine a psychological issue requiring primary attention amongst pain, anxiety, depression, and PTSD, the method may prioritize PTSD over all identified psychological issues of the user. Defining the psychological problem may further include transforming the result of a psychological evaluation test provided to the user or the user's account of the situation leading to the psychological issue/problem.

The psychological evaluation may include a set of questions as are well-known in the field, including but not limited to a written questionnaire, an audio/video-based questionnaire, and the like. It may include a customized questionnaire based on the questionnaire well-known in the field of psychological evaluation. Additionally, the psychological evaluation may include the user's medical history including, but not limited to, the user's vital sign report, such as pulse rate, temperature, respiration rate, blood pressure, and the like, which indicate the state of a user's or patient's essential bodily functions.

In accordance with an embodiment, the defining in Block 102 may be based on a first set of data received from the psychological evaluation tests, and/or a second set of data received from bio-signal measured through sensors applied on the user. The second input data which is data obtained from the sensors applied to the user may include sleeping information of the user, reference signal of the user's voice signal obtained from the acoustic sensor, and the like. The results obtained from at least one evaluation test, a medical history, and/or information obtained from the sensors are used to define the current state of mind of the user and identify a pattern leading to the psychological issue.

Based on the defined problem in Block 102, a target outcome is determined in Block 104 that is required by the user to overcome the identified problem in Block 102. The target outcome may be based upon target outcomes well known in the conventional art for the identified psychological issue. If the target outcome is not well defined in the known art, a therapist or a medical practitioner is presented with the defined problem of Block 102, to determine a target outcome needed for the user. In an alternate embodiment, the data and results obtained in Block 102 may be presented to the therapist or medical practitioner to further validate or modify the defined problem.

In Block 106, method 100 includes selecting at least one thought the user needs to overcome the problem based on the target outcome determined in Block 104. Said selection may be based on a thought process or pattern, as is well-known in various psychological evaluations, for achieving the determined target outcome in the present invention. Said thought may include at least one of a saying, a verse, a proverb, a poem, or a combination thereof that is well known to evoke a change in the state of mind defined in Block 102. In an alternate embodiment, the therapist or the medical practitioner may suggest a set of more psychologically healthy patterns, more insightful, of higher consciousness, more adaptive, wise, or psychologically aware patterns different from the selection provided in Block 106. In embodiments described below, AI and ML may assist and/or be used as a helping tool in lieu of the therapist or the medical practitioner.

In Block 108, method 100 includes having the user concentrate on the at least one thought selected in Block 108 to overcome the problem and achieve the target outcome, determined in Block 104, of the user. To make the user concentrate on the at least one thought, the method 100 may include suggesting a pre-determined schedule for the user to focus on the selected thought, preferably, in a relaxed, more meditative or contemplative way. The pre-determined schedule may include at least one of a predetermined time, a particular day, a duration, and the like, which the user needs to follow to achieve the target outcome identified in Block 104.

Based on the at least one thought selected in Block 106, at least one media that corresponds to the at least one selected thought is searched in Block 110. In an embodiment, the at least one media may include at least one song with lyrics and/or at least one video that corresponds to the at least one thought and is capable to evoke change in the state of mind leading to the psychological issue. Preferably, the at least one video, or live teaching to a group, is a didactic teaching that corresponds to the at least one thought that corresponds to both the old thought and while training in the new thought. The goal of the teaching is to evoke change in the state of mind and subconscious internal previous learning of the user from early childhood or later leading to the psychological issue.

The media may be selected entirely or partly from any known media databases. The said method 100 may also include providing the user with at least one media separate from the at least one thought selected in Block 106 or combining the at least one thought with the media into a single media file for providing the user. The media prescription may be considered to be a building of a series of custom, individualized content or music playlists for a user or therapy seeker experiencing a wide range of psychological issues, such as depression, sleep disorders, pain management, dementia, and so on.

In Block 112, the method 100 includes having the user anchor to the at least one media with the user achieving the target outcome. By anchoring, the method 100 provides the user or the patient a schedule either pre-determined based on known psychological therapy or customized by a medical practitioner based on user details such as the user's daily routine, demographic details, the intensity of the psychological issue, and the like, for following the suggested therapy. The anchoring preferably includes didactic teaching.

Lastly, the method 100 in Block 114 includes treating the user by having the user play the media multiple times to achieve the targeted outcome. Treating may include providing the user with the selected media and at least one thought selected in Block 106 along with selecting at least one media in Block 110 either separately or incorporated into a single file. In an embodiment, the treatment may include providing the user with a schedule for adherence to the suggested therapy to achieve the target outcome determined in Block 104. Treating the users/patients with specific psychological issues through media is referred to as the “non-media outcome’ to be attained. In a preferred embodiment, the user learns through the didactic teaching of the trained professional paired with the selected media or the theme of the selected media.

In an alternate embodiment, the treatment may further include monitoring adherence to the suggested therapy. The step of monitoring may include providing the user with a supplemental evaluation test or a self-evaluation test at a pre-determined interval.

In accordance with a further embodiment, the method 100 may include the trained professional creating a video (or teaching live to a group or individual) of the selected at least one media (or specific theme within that media) where the at least one media is a song with lyrics and treating the patient by having the patient play the video multiple times, journaling, taking their own notes, asking themselves the question as to how to apply didactic trainings to their own individual lives to achieve the targeted outcome.

In another embodiment, the method 100 may include determining effectiveness of the at least one media in achieving the target outcome and changing the at least one media. For the determination, method 100 may be based on a report of the evaluation test to determine the effectiveness of the suggested therapy and accordingly, modify the therapy including the at least one thought and/or at least one media selected earlier.

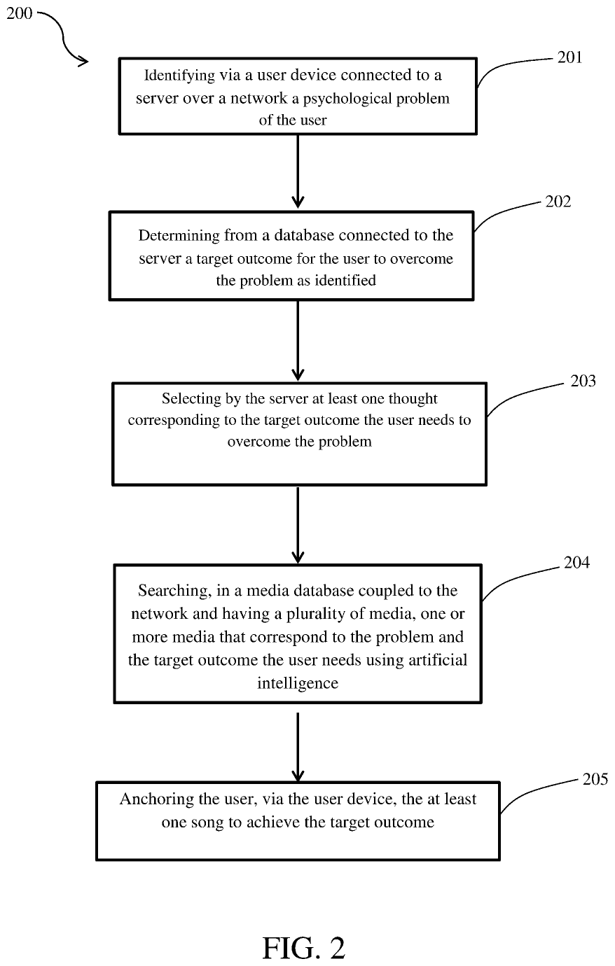

In accordance with an alternate aspect of the present invention, FIG. 2 illustrates a method 200 flow chart illustrating psychological health management. In one embodiment, the present disclosure provides a computer-implemented method 200 for measuring a psychological state of a user, wherein the method is implemented using a computer device having an input component and executing at least one client application, which may in turn include a plurality of client applications. In some embodiments, the computer device is selected from the group consisting of a desktop computer, a laptop computer, a computer tablet device, a smartphone device, and the like. In some embodiments, the computer input device is selected from the group consisting of a mouse component, a stylus computer, a keyboard component, a microphone, and a sensor to sense the physical state of the user (e.g., EEG, thermometer, infrared scan, heart rate or pulse sensor, accelerometer, and/or gyroscope and the like), and a touch screen display. It is appreciated that many such computer input methods are available, and advances in computing technology will continue to provide new types of inputs. The method of the current patent is dependent on an input modality, but importantly is independent of a specific type of input modality so long as the ability to reliably measure the input is maintained, and thus the disclosed methods are applicable to current and future input modes.

In an embodiment, the computer-implemented method 200 for providing customized therapy media to a user based on the psychological state of the user, the method 200 comprising: identifying in Block 201, via a user device connected to a server over a network, a psychological problem of the user; determining in Block 202, from a database connected to the server, a target outcome for the user to overcome the problem as identified; selecting in Block 203, by the server, at least one thought corresponding to the target outcome the user needs to overcome the problem; searching in Block 204, in a media database coupled to the network and having a plurality of media, one or more media that correspond to the problem and the target outcome the user needs using artificial intelligence; and anchoring the user in Block 205, via the user device, to at least one media from said one or more media to achieve the target outcome.

In accordance with an embodiment, the computer-implemented method 200 may include a client application of the PMS installed in the user devices and containing at least one application configured to be executed by the user device, or a computer-readable recording medium on which the computer program product is recorded. The identifying in Block 201 may include at least one such application configured to perform a method of determining a state of mind according to the present invention, for example, to obtain data about the psychological state of the user. As an example, the application may be configured to provide an evaluation test, such as a written questionnaire or an audio/video questionnaire, to receive a response from the user.

The application may also be configured to receive input data from a plurality of sensors attached to a person of the user, to capture for example vital signs of the user. The data may also be used as supplemental data to the evaluation test. The identification of the psychological problem may also include user details, such as, but not limited to, the user's profile, user's medical history (psychological and physical), the user's demographic details, and other such details that may aid in the identification of the psychological problem of the user.

In another aspect of the present invention, the client application installed on the user devices may be configured to communicate to the server via any web browser known in the art to access a web-based psychological evaluation test. The user may respond to the evaluation test, e.g., prompts, by entering or selecting responses to the questionnaire into the device and transmitting the responses over the network to the PMS server, thus interactively communicating remotely with the server. The evaluation test may be adjusted based on the user's details concerning the medical history of the user.

In an alternate embodiment, the identification in Block 201 may receive conversation content or video content from the user devices to the PMS server. The computer-implemented method 200 may further include identifying, using artificial intelligence (AI), the psychological problem of the user based on the shared content by the user. For example, the content shared by the user includes data in audio format inputted through a microphone, describing the psychological state of the user's mind is transmitted to the PMS server. The PMS server may use this shared data for psychological health assessment and/or psychological illness treatment, using AI.

In one embodiment, the computer-implement method 200 can automatically convert the user entered data into language and terminology that complies the medical, psychological, and scientific databases which includes journal articles, lectures, and textbooks. In another embodiment, the computer data that complies with medical, psychological, and scientific databases can be automatically converted to popular or modern language patterns to enable searching of media including songs and videos.

In accordance with an embodiment, the identification of psychological issues or conditions may be based on responses to the evaluation test. Based on the responses, the computer-implemented method 200 determines at least one thought and corresponding media content to the user to address the psychological condition affecting the user. In other embodiments, the psychological evaluation test may be composite measures based on responses to one or more evaluation tasks and/or bio-signals received from sensors. In some embodiments, the psychological evaluation may be based on a composite evaluation computed using additional external or non-performance information such as the user's demographic information or normative data and the like.

In an embodiment, the computer-implemented method 200 may be configured to rank or prioritize a user's psychological conditions, such as based on a known relationship between various psychological conditions and the intensities of these conditions as determined from various psychological tests conducted on the patient. For instance, to determine the psychological issue requiring primary attention amongst pain, anxiety, depression, and PTSD, the method 200 may prioritize PTSD over all identified psychological issues of the user.

In Block 202, the computer-implemented method 200 determines the target outcome based on the responses or data received in Block 201. For this purpose, the server receives the data from the user devices and compares it with a database containing a set of known psychological issues. The database can include but is not limited to the Clinical Handbook of Psychological Disorders. An in-depth evaluation is performed so that the server can judge or determine the psychological state of the user with reference to the obtained self-diagnosis response data and judge a suitable target outcome the user needs to overcome the identified problem in Block 202. Specifically, if it is determined in Block 202 that the user's psychological state corresponds to a known psychological condition, the server assists the user by sending a target outcome that the user needs to achieve.

In Block 202, determining the target outcome may also include the user's vital signs such as heart rate, respiration rate, body temperature, skin temperature, measurements related to restlessness, measurements related to sleep quality, measurements related to attention level, measurements related to concentration level and so on. Sensors can be used to detect, measure and log or map the user's vital signs throughout the entire process. The vital signs may also include a change in breathing pattern, change in eye contact, and the like. Any type of measurement or quantifiable data schedule associated with a patient may be considered a patient vital sign useful in assessing the user and/or modifying treatment.

In Block 203, the server selects at least one thought corresponding to the target outcome, determined in Block 202, the user needs to overcome the problem identified in Block 201. As illustrated in FIG. 2, the server may include a plurality of known thoughts associated with the determined target outcome that has been ascertained by known psychological evaluation. In an alternate embodiment, the server may be interlocked with a separate database having a plurality of known thoughts to overcome the target outcome.

The server may also employ an artificial intelligence system to identify the at least one thought. Alternatively, the server may provide the user with a plurality of thoughts which may depend upon the intensity of the psychological issue of the user and the target outcome. In some embodiment, the server may send the determined target outcome in Block 202 and the selected at least one thought to a medical practitioner for further evaluation and modification.

As illustrated in FIG. 2, in Block 204, the method 200 includes searching, in a media database coupled to the network and having a plurality of media, one or more media that correspond to the problem and the target outcome the user needs using artificial intelligence. For instance, the server may be configured to analyze and characterize media stored in a database, which may be broadly defined as any sound/video, sequence of sounds/video, and/or portion thereof.

The term media as used herein is intended to broadly denote any of a musical song, a voice presentation such as a lecture or sermon, a natural sound recording, an artificially generated sound recording, a video representing a song, an artificially generated video and/or any other audible information. In some embodiments, the server may use artificial intelligence (AI), machine learning (ML), and/or other techniques to break down media into constituent feature vectors or other representations to assign the media to specific charactered clusters based on dimensionality components, key characteristics, and/or other factors. The server may select a plurality of therapeutic media by arranging specific media (audio/video segments or portions thereof) in a specific order based on the target outcome and the corresponding thought selected for a particular user or patient. These AI and ML embodiments are described in more detail below.

The server may be used to characterize each media stored in the media database in accordance with various features extracted via analysis of the media or portions thereof to create a database of media features. The media features are descriptive of the media or portion(s) thereof. Media components or features of interest may include genre, vocalization including voice tones, instrumentation, timbral brightness, clarity, rhythm, tempo (e.g., beats per minute), time spent in major keys, texture, pulse strength, lyrical sentiment, and so on. Many different features may be used to analyze and characterize media (i.e., audio/video information). The method 200 may be configured to update the media database in response to general research of individual patient data about the physiological impact of media on one's brain, the application of media therapy best practices, the use of key elements in songs to create therapy designed to evoke a desired outcome in a patient, and optionally the use of patient-relevant faith-based content to further the desired outcome.

The method 200, in Block 205, comprises anchoring the user, via the user device and preferably, the didactic teaching of the trained professional, to the at least one song to achieve the target outcome. By anchoring, the method 200 provides the user with a schedule, either pre-determined based on known psychological therapy or customized by a medical practitioner or user based on user details such as the user's daily routine, demographic details, the intensity of the psychological issue, and the like, for following the suggested therapy.

The anchoring, may include playing the at least one media and at least one thought multiple times in a loop. The loop ought to be continued until the target outcome is achieved or in a case where achieving the target outcome is delayed after a predetermined time, changing the thought the user needs to overcome the problem. Preferably, the anchoring is performed after entering into, listening, journaling, and applying the didactic teaching of the professional therapist.

Anchoring may further comprise receiving from the user device a compliance report on meditating, contemplating, and journaling along with listening to the at least one media by automatically acquiring a daily report. Said compliance may be requested from the user via the user device or simply be a report from the device after a pre-determined time. Such a report may include the number of times the media and/or the teaching was played, the play duration, the average playtime, the time, and day when the media was played, and so on. The said media prescription, i.e. at least one thought and at least one corresponding media, is further evaluated based on said report and the effectiveness of the suggested therapy is determined.

The above method can be executed by a computer system (described below) to identify various psychological issues or conditions affecting a user.

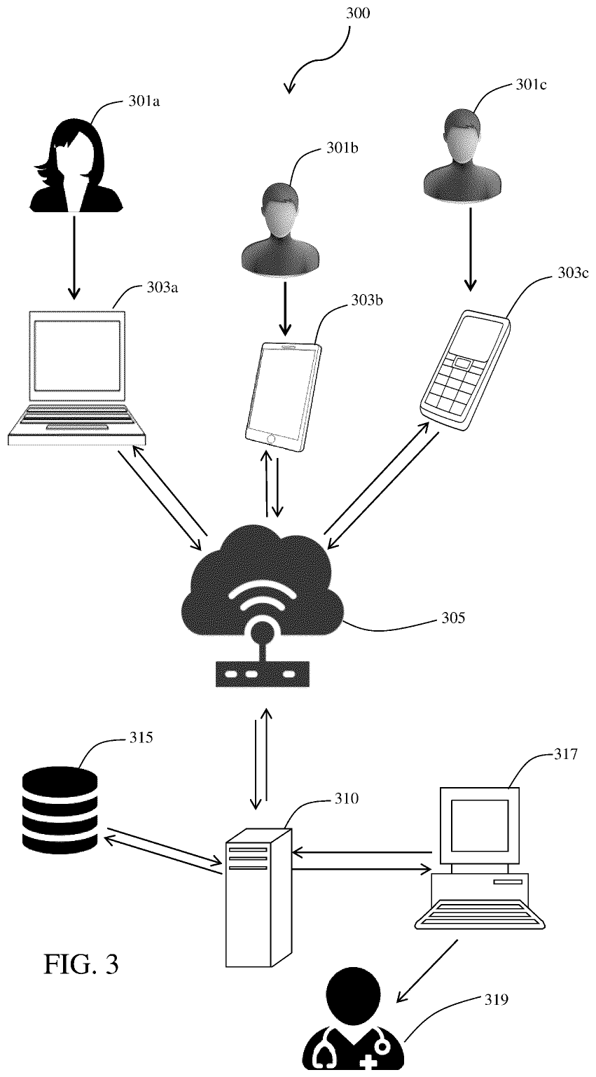

FIG. 3 illustrates an exemplary network architecture 300 for implementing a psychological health management system, in accordance with the present invention. In one embodiment, the network architecture 300 implementing the psychological health management system (PMS) includes at least one of user devices 303a-303c, collectively referred to as user devices 303, and a server 310 configured to communicate with each other via one or more communication networks 305. Furthermore, the PMS hosted on the server 310 may include at least one network communication unit 305, a set of input/output devices, a set of local data storage device, such as disk drives, and at least one memory in which data and program instruction sets executable by one or more processing units, including operating system, can reside. In accordance with an embodiment, the network architecture 300 may include at least one media database 315 connected to the server 310 via the communication network 305 or an alternative communication network. The server 310 may include a therapist/medical practitioner device(s) 317 connected either via the communication network 305 or an alternate communication network and is configured to display the (anonymous, encrypted) details of the patients or users 301a-301c, collectively referred to as users 301, the psychological assessment tests and corresponding responses, the assigned psychological therapy and such other details required by the therapists/medical practitioner 319 to determine the effectiveness of the therapy.

User devices 303 may include mobile devices, desktop devices, laptops or any other type of computing devices known in the art, having both input elements and output elements. Input elements may include a touch screen display, keyboard, input buttons, audio input devices, video input devices and the like that allow the users 301, to enter information into the user devices 303. In a further embodiment, input elements may also include sensors, such as EEG sensors, an oximeter, a glucometer, infrared sensors, heart rate sensors, an electrocardiogram sensor, and the like that are configured to monitor users' physical state and provide the data as input to user devices 303. The output elements may include a display unit, a speaker unit, a projector unit, and the like, which are capable to provide the user with prompts and/or feedback sent by the PMS server 310.

The user devices 303 may download a client application of the PMS and present the same, via a graphical user interface or GUI, to the users 301 to gain access to the server 310 implementing the PMS via respective user devices 303. Said client application of PMS may also include an oral/written evaluation test configured to receive responses from the users to define the psychological state of the users 301. Alternatively, the users 301 may access, via their user devices 303, a web-based evaluation test hosted on the PMS server 310 by using any web browser known in the arts. The evaluation test may be any known questionnaire in such treatments, similar psychological assessment tests or a customized version of such known questionnaire. Said assessment may also include an audio/video evaluation test. Additionally, the client application of the PMS may include data from sensors, such as EEG and the like, to supplement the evaluation test. Apart from the response data, the user devices 303 may be configured to collect and send other user details, such as anonymous or professional user profiles, demographic detail, and/or medical history to the PMS server 310.

In one aspect of the invention, the PMS server 310 is generally comprised of one or more computers, each of which is equipped with at least one microprocessor. The number of computers and processors will typically be determined by the required processing capacity of the system 300, which, in turn, depends on the projected workload. The workload is defined by the number of users 301 who have access to the server 310 and the volume of data that needs to be processed.

In certain embodiments, a third-party cloud computing platform might be utilized for the server 310, allowing the allocation and modification of physical hardware resources dynamically in response to demand. However, to simplify the subsequent description, we assume that the exemplary PMS server 310 consists of a single computer housing a solitary microprocessor. In the presently described embodiment, the program instructions include instructions implementing communications with one or more client applications via one or more user devices 303, such as an application executing on a smartphone 303c, desktop PC 303a, tablet 303b, or other device operated by the users 301 or a supervising medical practitioner 319. These communications operations enable data of the users or therapy seeker 301, to be received for processing by the PMS server 310.

The server 310 side-implementation of the PMS receives at least one response from the user 301, via the user devices 303 over the communication network 305. Such response may be provided to the server 310 via one or more input devices available with the user devices 303. For example, when a user 301 accesses the web-based self-diagnosis evaluation test stored on the server 310 using a user device 303 such as a smart phone 303c, a PC 303a, or a tablet 202b, the web-based evaluation test may include a plurality of discrete audible and/or visual segments such as questions to be responded to by the user 301 being evaluated. The server 310 communicates with the audible playback means and recording means, such as a microphone of the user devices 303, and records the responses of the users 301. Alternatively, the user 301 may choose to provide inputs by typing the response through an input device (for example, a keyboard, a mouse, or a touch input). In some embodiments, the user devices 303 may include a network interface, such as a Wi-Fi interface or a cellular mobile interface including, e.g., a Nano Sim card, enabling it to connect and transfer data directly to the PMS server 310 via the communication network 305. The server 310 acquires and stores the response data for further evaluation. In an alternate embodiment, the user devices may be configured to store a local copy of the user responses to the evaluation test.

The server 310 is configured to implement the PMS that defines the psychological problem based on the response data and other data and determines a target outcome, at least one thought based on the target outcome, and selects at least one media corresponding to the at least one thought. The server 310 further determines the effectiveness of the assigned psychological therapy, wherein such determination may include the effectiveness of the at least one thought and/or the effectiveness of the at least one media assigned with the psychological therapy. In order to determine the effectiveness of the therapy, server 310 may be configured to provide a second evaluation test to the user 301 at a pre-determined time interval or upon completion of the designated therapy. In a further embodiment, the determination of effectiveness, system 300 may identify changes in responses to the second evaluation test and compare the first data of the first evaluation test. The system 300 may also be configured to obtain data from sensors worn by the patient during the process of assigned psychological therapy to assign a quantitative or qualitative score indicative of the user's psychological condition(s). Based on the effectiveness of the therapy or if the system identifies a different psychological issue, the system may be configured to modify or change the assigned psychological therapy, which may include changing or modifying the at least one different thought and/or the at least one media to address the identified psychological issue.

While all sensors can be utilized to detect and or record a user's state of mind, medical condition, or psychological state, the preferred method is to use noninvasive sensors. The noninvasive sensors include but are not limited to Electrocardiogram (ECG/EKG) Sensors, Photoplethysmogram (PPG) Sensors, Electroencephalogram (EEG) Sensors, Galvanic Skin Response (GSR) Sensors, Respiration Rate Sensors, Temperature Sensors, Accelerometers, Near-Infrared Spectroscopy (NIRS) Sensors, Eye-Tracking Sensors, and combination thereof.

ECG/EKG sensors measure the electrical activity of the heart and can provide information on heart rate, heart rhythm, and overall cardiovascular health. PPG sensors utilize light to measure blood volume changes in peripheral blood vessels. They can provide information on heart rate, blood oxygen saturation levels, and pulse waveform analysis.

EEG sensors measure the electrical activity of the brain and can provide insights into brainwave patterns, sleep stages, and cognitive states. GSR sensors measure changes in skin conductance, which can be indicative of emotional arousal or stress levels. Respiration Rate sensors monitor the rate and depth of a person's breathing, providing information on respiratory health and patterns.

Non-invasive temperature sensors measure body temperature and detect fluctuations that may indicate fever or other health conditions. Accelerometers measure movement, acceleration, and orientation of the body. They can provide information on physical activity levels, posture, and movement patterns. NIRS sensors measure changes in oxygen levels in the blood and can provide insights into brain activity, tissue oxygenation, and cerebral blood flow. Eye-tracking sensors track eye movements and gaze patterns, offering insights into attention, cognitive processes, and visual perception. Voice Analysis sensors can capture and analyze speech patterns, voice quality, and vocal biomarkers to assess psychological states, such as stress or emotional well-being. Non-invasive blood pressure monitors utilize cuffs or optical methods to measure blood pressure, providing information on systolic and diastolic pressure. Pulse oximeters measure blood oxygen saturation levels and pulse rate, often through a clip-on sensor on a finger or earlobe. Force plates measure ground reaction forces and balance distribution, providing insights into posture, stability, and gait analysis. Infrared Thermography or infrared cameras capture temperature variations on the skin's surface, which can indicate inflammation, circulation issues, or localized temperature changes.

While these sensors, listed above, are considered non-invasive, their accuracy and capabilities may vary. Proper calibration, validation, and adherence to best practices are necessary for accurate and reliable measurements. Additionally, specific regulatory and safety considerations may apply when using these sensors for medical or psychological applications.

In another embodiment, the network architecture 300 includes at least one media database 315 in communication to the server via at least one communication network via a wired or wireless network. The media database 315 is configured to provide media content from the data storage unit(s) to the server 310. The media database 315 may also be configured to store user details including, but not limited to, user personal info and medical details, therapist details, analysis reports, therapy progress and/or search results of each of the users 301 received from the server 310.

Said communication network 305 may include, but is not limited to, the Internet, a local area network (LAN), a wide area network (WAN), a satellite network, and/or a cellular network. Communication network 305 may include a wired or wireless network asset, such as but not limited to a local area network (LAN), a wide area network (WAN), a wireless personal area network (PAN), and the like. Similarly, computing devices 303, 310, 315, and 317 may be connected over the internet, an intranet, an extranet, an Ethernet, or any other system that provides communication. Further, the computing devices 303, 315, and 317 may include at least one processor and one or more applications executed on the processor capable of communication with the PMS server 310.

In one embodiment, system 300 includes a therapist computer device 317 in communication with the server 310 via the communication network. The computer device 317 is configured to output, to a therapist or medical practitioner 319, the user data, including user profile, user medical history, the evaluation test and its responses, sensor data, and such data required by the medical practitioner 319 to evaluate the effectiveness of the assigned therapy. The computer device 317 may also be configured to provide two-way communication between the user 301 and the medical practitioner 319. The medical practitioner 319 may modify or change the assigned therapist or prompt the user 301 for a second evaluation test. This embodiment enables the computer to have additional functionalities including the ability to work seamlessly with both the user and the medical professional and allow both the user and medical professional the ability to complete evaluations quicker, as described below.

In some embodiments, the system 300 also includes a knowledge base, which contains information generated via machine learning methodologies, using data obtained via expert evaluation of one or more training sets of subjects, and embodying a computational model of a relationship between mental state, e.g., subject's mental health and heart rate characteristics. Various machine-learning methodologies may be employed in different embodiments of the invention, including, but not limited to, decision tree learning; association rule learning; artificial neural networks; inductive logic programming; support vector machines; cluster analysis; Bayesian networks; reinforcement learning; representation learning; similarity learning; sparse dictionary learning; and/or genetic algorithms.

The knowledge base may be contained within the non-volatile storage or may be stored in a separate storage device, which may be directly connected to the PMS server 310, or may be remotely located. Since the knowledge base may ultimately grow to contain very large amounts of training and historical subject data, it may be advantageous for the knowledge base to be stored in a large data center and/or one or more distributed databases, e.g., in a cloud storage service. The exact form and location of the knowledge base are not critical, so long as the required data, as described below, is accessible for processing by the PMS server 310.

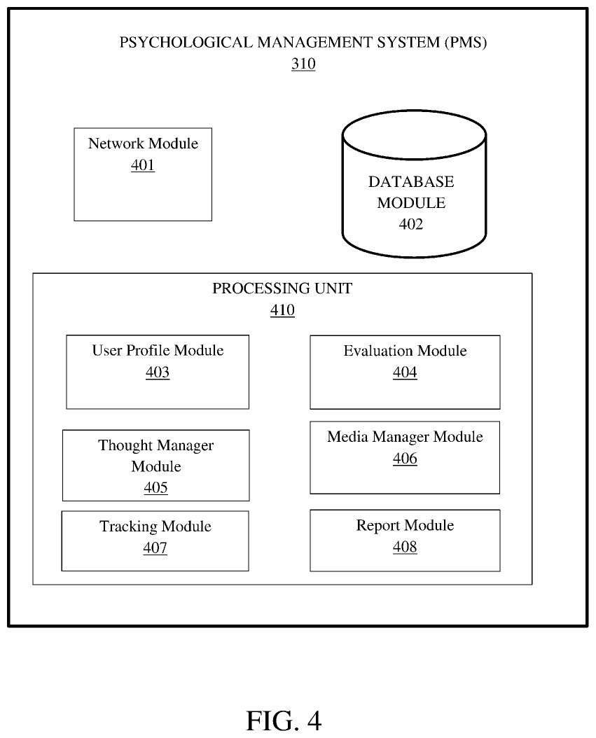

FIG. 4 illustrates the psychological management system (PMS) 310 of FIG. 3. In an embodiment, the PMS 310 may include one or more computers, each of which includes at least one processing unit 410. The number of computers and processing 410 will generally depend upon the required processing capacity of the system 310, which in turn depends upon the anticipated workload, i.e., the number of patients or therapy seekers having access to the platform 310, and the volume of data to be processed. In further embodiments, a third-party cloud-computing platform may be employed for platform 310, thereby enabling the physical hardware resources to be allocated, and changed, dynamically in response to demand. However, for simplicity in the remainder of the description, it is assumed that the exemplary PMS 310 includes a single computer with a single processing unit 310.

The processing unit 410 is connected to or otherwise linked with a non-volatile memory/database module 402. The non-volatile database module 402 could be a hard disk drive or may incorporate a solid-state non-volatile memory, such as read-only memory (ROM), flash memory, or similar technology. Additionally, the processing unit 410 may be connected to volatile storage (not depicted here), such as random access memory (RAM), which stores program instructions and temporary data relevant to the functioning of the PMS 310.

In a typical configuration, the storage device 402 contains operating system programs, data, and other executable application software essential to the intended operations of the PMS 310. Furthermore, in these embodiments, the database module 402 may also store program instructions that, when executed by the processing unit 410, enable the PMS 310 to carry out operations for the implementation of a psychological assessment and therapy method, as described in FIG. 2. During operation, instructions and data stored on storage device 402 are transferred to volatile memory as needed and executed accordingly.

The processing unit 410 is also operably associated with a network module 401 conventionally. The network module 401 facilitates access to one or more data communications networks, such as the Internet, employed for communication between platform 310 and the user devices 303. In use, the processing unit 410 includes various other modules, such as a user profile module 403, an evaluation module 404, a thought manager module 405, a media manager module 406, a tracking module 407, and a report module 408, having program instructions configured to perform processing and operations embodying features of the present invention, comprising various steps in the method described above with reference to the flowcharts, data structures, and software architectures illustrated in FIGS. 1 to 3.

The program instructions contained in said modules may further include instructions embodying a web server application. Data stored in the non-volatile and volatile storage may then include web-based code for presentation and/or execution on user devices (e.g. HTML or JavaScript) facilitating a web-based interface to the PMS. The web-based interface may, for example, enable the upload of data from any device, including smartphone 303c or desktop PC 303a, to the PMS server 310. The web interface may also enable the users 301 and/or their health care professional 319, via devices 303 and/or 317, to access data that has been stored and processed by the PMS server 310.

A method to store, convert, transmit, and display the patient's information is provided and can be incorporated into his process, as needed. This information in this method includes but is not limited to the patient's self-described state, thought pattern, diagnosis, preferred or more empowered thought pattern, history, and combinations thereof. In one embodiment this methods comprises a) storing information in a standardized format about a patient's condition in a plurality of network-based non-transitory storage devices having a collection of medical records stored thereon; b) providing remote access to users over a network so any one of the users can update the information about the patient's condition in the collection of medical records in real time through a graphical user interface, wherein the one of the users provides the updated information in a non-standardized format dependent on the hardware and software platform used by the one of the users; c) converting, by a content server, the non-standardized updated information into the standardized medical, scientific or psychological format, d) storing the standardized updated information about the patient's condition in the collection of medical records in the standardized format; e) automatically generating a message containing the updated information about the patient's condition by the content server whenever updated information has been stored or is needed; and f) transmitting the message to all of the users over the computer network in real time, so that each user has immediate access to up-to-date patient information.

An additional embodiment would include storing information about a patient's condition in a plurality of network-based non-transitory storage devices having a collection of medical records stored thereon; b) providing access, by a content server, to users so that any one of the users can update the information about the patient's condition in the collection of medical records, and; c) storing the updated information about the patient's condition in the collection of medical records in the plurality of network-based non-transitory storage devices. Users can include one or more patients and one or more medical practitioners or experts assisting the patient or medical practitioners. These methods to store, convert, transmit, and display the patient's information can be used in the entirety as described above or piecemeal, as needed.

In an alternate embodiment, the PMS may include a data learning module. Conversation text such as an audio or text that expresses the psychological state of the user may be used by the data learning module to identify the psychological problem of the user. The data learning module may identify the psychological problem based on a response to the user's conversation from predetermined data by using the learned data learning model. The data learning module acquires predetermined data according to a predetermined criterion by learning and uses the data learning model using the acquired data as an input value, thereby identifying the psychological state based on the predetermined data. In addition, a result value output by the data learning model using the acquired data as an input value may be used to update the data learning model.

The data learning model may be constructed in consideration of the application field of the learning model, the purpose of learning, or the computer performance of the device. The data learning model may be, for example, a model based on a neural network. For example, models such as Deep Neural Networks (DNN), Recurrent Neural Networks (RNN), Long Short-Term Memory models (LSTM), BRDNN (Bidirectional Recurrent Deep Neural Networks), and Convolutional Neural Networks (CNN) are used as data learning models.

Embodiments described herein, particularly with reference to FIGS. 1 to 4, employ techniques including metric learning and decision tree learning. However, these approaches should be regarded as illustrative only, and do not exclude the use of other learning techniques and computational models from the scope of the invention.

Some embodiments will be described within the context of a system for providing therapeutic content such as music to residents or patients within the context of an assisted living/managed care environment, hospital, or other institution (medical or nonmedical). However, those skilled in the art and informed by the teachings herein will realize that the invention is also applicable to other technical areas and/or embodiments. For example, embodiments of the invention have use case applicability within the context of schools, prisons, hospitals, and other (typically) institutional settings where music or content-based therapy can be delivered to patients. The methodology provides the flexibility to respect the free volition of a test subject person, a patient, or a prisoner to listen to said media by giving the patient multiple choices including the choice to opt-out.

In one embodiment, the present invention provides a method for treating the psychological issues of a user through the use of AI and ML techniques to provide customized therapy media. The method involves several steps that leverage AI capabilities to improve the overall effectiveness of the therapy process

Step (a): Defining the user's problem: The method starts by defining the psychological problem experienced by the user. AI algorithms analyze user input, such as verbal or written descriptions, to determine the specific nature of the problem. The AI can convert the language of the user into precise scientific or psychological language that can be used to search the scientific, medical, and psychological journals and handbooks to determine the illness or condition. Over time, this process can be improved with ML.

Step (b): Determining the target outcome: Based on the identified problem, the AI system determines a target outcome that the user needs to achieve to overcome the problem. ML algorithms analyze historical treatment data to suggest appropriate target outcomes based on similar cases.

Step (c): Selecting relevant thoughts: Using ML techniques, the AI system selects one or more thoughts or beliefs that the user needs to overcome in order to achieve the target outcome. This selection is based on a combination of the user's psychological state, problem definition, and desired outcome.

Step (d): Concentration on selected thoughts: The user is prompted to concentrate on the selected thoughts or beliefs identified in step (c). The AI system provides guidance and support to help the user focus on these thoughts, facilitating cognitive restructuring and reframing.

Step (e): Searching for appropriate therapy media: The AI system employs ML algorithms to search for therapy media, such as songs with lyrics or videos, that align with the selected thoughts and the target outcome. The search is based on the semantic analysis of media content and user preferences obtained through user feedback or historical data

Step (f): Anchoring to the therapy media: The user is encouraged to anchor or associate themselves with the selected therapy media. By repeatedly engaging with the media, the user strengthens the connection between the media content and the desired target outcome, reinforcing positive associations.

Step (g): Treating the user with media exposure:

The AI system guides the user to play the selected therapy media multiple times to achieve the targeted outcome. The frequency and timing of media exposure can be personalized based on user progress, engagement levels, and real-time emotional states tracked by AI algorithms.

By incorporating AI and ML techniques, embodiments of the present invention offer several advantages over traditional psychological therapy methods. The AI system assesses the user's psychological state, tailors therapy media to match specific thoughts and desired outcomes, and adapts the treatment based on user feedback and progress. This personalized and data-driven approach increases the effectiveness and efficiency of psychological treatment, leading to improved user outcomes and satisfaction.

These embodiments harness the power of AI and ML to revolutionize psychological treatment by delivering customized therapy media tailored to the unique needs of users. By leveraging AI algorithms for assessment, selection, and personalization, the method significantly enhances the therapeutic process, facilitating the achievement of target outcomes and ultimately improving the overall effectiveness of psychological treatment.

In one embodiment, the present invention introduces a computer-implemented method for providing customized therapy media to users based on their psychological state. By utilizing AI algorithms, network-connected user devices, and a media database, the method delivers personalized therapy content to users efficiently and effectively.

Step (a): Identifying the user's psychological problem: The method begins by utilizing a user device connected to a server over a network to identify the user's psychological problem. The user device captures user input, such as self-reported descriptions or responses to assessment questionnaires, which are then transmitted to the server for analysis.

Step (b): Determining the target outcome: Based on the identified psychological problem, the server accesses a database connected to it to determine a target outcome that the user needs to achieve in order to overcome the problem. The database contains historical treatment data, expert knowledge, and ML models that aid in suggesting appropriate target outcomes based on similar cases

Step (c): Selecting relevant thoughts: The server, employing AI techniques, selects one or more thoughts or beliefs that correspond to the target outcome the user needs to overcome the identified problem. The selection is based on a combination of the user's psychological state, problem definition, and desired outcome.

Step (d): Searching for appropriate therapy media: Using AI algorithms, the server searches a media database coupled to the network that contains a wide range of media content. The search aims to find one or more media that correspond to the identified problem and the target outcome. The AI algorithms employ semantic analysis, sentiment analysis, and user preferences to identify relevant media that align with the user's needs.

Step (e): Anchoring the user to selected media: The server communicates with the user device to anchor the user to at least one selected media from the search results. Through the user device, the selected media, which could be songs with lyrics, videos, or other forms of media, are presented to the user. The user engages with the media, repeatedly listening or interacting with it to strengthen the association or connection between the media content and the desired target outcome.

This embodiment offers several advantages over traditional therapy methods. By utilizing network-connected user devices and AI algorithms, the method provides a personalized and accessible approach to therapy delivery. Users can easily access therapy media via their devices, and the AI-driven selection process ensures that the media aligns with their psychological state and desired outcomes. Additionally, the method benefits from the scalability of network connectivity and the efficiency of AI techniques, resulting in improved treatment outcomes and increased user satisfaction.

Utilizing this embodiment, AI techniques and network-connected user devices can provide customized therapy media to users based on their psychological state. By leveraging AI algorithms for problem identification, target outcome determination, thought selection, media searching, and user anchoring, the method enhances the delivery of personalized therapy content, improving the effectiveness and accessibility of psychological treatment.

In one embodiment, the present invention introduces a system for providing customized therapy media to users based on their psychological state. By employing artificial intelligence algorithms, a non-transitory storage medium, and a processor, the system facilitates personalized therapy delivery.

The system comprises a non-transitory storage medium and a processor coupled to the storage medium. The storage medium stores databases containing user data, therapy resources, media content, and AI models for processing and analysis. The processor is configured to execute the various functions of the system, including problem identification, target outcome determination, thought selection, media searching, and user anchoring.

In one embodiment, the system operates using 5 steps.

Step (a): Identifying the user's psychological problem: The system, via the user device, identifies the user's psychological problem using assessment tools, user input, or other means. The processor accesses the stored databases to analyze the data and determine the specific nature of the problem.

Step (b): Determining the target outcome: Based on the identified psychological problem, the processor accesses a database connected to the server to determine a target outcome that the user needs to achieve to overcome the problem. The database contains historical treatment data, expert knowledge, and AI models that aid in suggesting appropriate target outcomes based on similar cases.

Step (c): Selecting relevant thoughts: Using AI techniques, the processor selects one or more thoughts or beliefs that correspond to the target outcome the user needs to overcome the identified problem. The selection is based on a combination of the user's psychological state, problem definition, and desired outcome.

Step (d): Searching for appropriate therapy media: The processor, utilizing AI algorithms, searches a media database coupled to the network that contains a variety of media content. The search aims to find one or more media that correspond to the identified problem and the target outcome. The AI algorithms employ semantic analysis, sentiment analysis, and user preferences to identify relevant media that align with the user's needs.

Step (e): Anchoring the user to selected media: The system anchors the user to at least one selected media from the search results. This anchoring is achieved through the collaboration between a trained therapist, who provides didactic teaching, and the user device, which presents the selected media to the user. The user engages with the media, repeatedly consuming or interacting with it to strengthen the association between the media content and the desired target outcome. The patient can then go home and repeat listening to the song, to reinforce and provide greater lasting change.

This embodiment offers numerous advantages over traditional therapy systems. By incorporating AI techniques, a non-transitory storage medium, and a processor, the system provides personalized and adaptive therapy delivery. The collaboration between a trained therapist and a user device ensures the effective transmission of therapy media to the user. This personalized and interactive approach enhances the therapy experience, leading to improved treatment outcomes and increased user satisfaction.

In this embodiment, the present invention introduces a system that utilizes artificial intelligence algorithms, a non-transitory storage medium, and a processor to provide customized therapy media to users based on their psychological state. By combining the expertise of a trained therapist with the technological capabilities of a user device, the system delivers personalized therapy content efficiently and effectively, enhancing the overall therapy experience for users.

Another embodiment further incorporates machine learning techniques to enhance the effectiveness and personalization of the therapy media selection process. The AI algorithm employed in step (e) and step (d) utilizes machine learning models that continuously learn from user feedback, user engagement patterns, and the analysis of treatment outcomes. By leveraging machine learning, the system improves its ability to recommend therapy media that aligns with the user's psychological state, preferences, and treatment goals.

The machine learning techniques employed in the AI algorithm employ various approaches, including supervised learning, unsupervised learning, and reinforcement learning. Through supervised learning, the system utilizes labeled data from previous therapy sessions, user ratings, or expert evaluations to train the model and improve the accuracy of therapy media recommendations. Unsupervised learning is employed to discover latent patterns and associations within the media database, enabling the system to identify relevant therapy media that may not have been explicitly labeled.

Furthermore, reinforcement learning techniques are utilized to adaptively refine the therapy media selection process based on real-time user feedback and treatment progress. The system learns from the user's responses and adjusts the recommendations to optimize the achievement of target outcomes. By continuously updating and fine-tuning the machine learning models, the system ensures that the therapy media delivered remains relevant, engaging, and effective for each user's specific psychological needs.

In an additional embodiment, the AI algorithm employed in steps (b) and (c) utilizes natural language processing techniques to analyze and interpret user input, including verbal or written descriptions, to identify the psychological problem, determine the target outcome, and select relevant thoughts for therapy.

The method/system according to claim 2 incorporates natural language processing (NLP) techniques to analyze and interpret user input, enabling a more comprehensive understanding of the user's psychological problem and therapy needs. The AI algorithm employed in step (b) and step (c) utilizes NLP techniques to process verbal or written descriptions provided by the user and extract meaningful information.

The NLP techniques employed in the AI algorithm include text classification, sentiment analysis, entity recognition, and semantic analysis. Through text classification, the system categorizes user input into relevant psychological problem domains, allowing for a more accurate determination of the target outcome. Sentiment analysis helps assess the emotional tone and intensity of the user's input, providing further insights into the user's psychological state.

Entity recognition techniques enable the identification and extraction of key concepts or keywords from the user's input, which are then used to select relevant thoughts or beliefs for therapy. The semantic analysis component of the AI algorithm allows for a deeper understanding of the user's input, identifying semantic relationships between words or phrases to ensure the selection of thoughts that are closely aligned with the target outcome and problem identification.

By incorporating NLP techniques into the AI algorithm, the system improves its ability to analyze user input and provide a more personalized and accurate selection of therapy thoughts and target outcomes, enhancing the overall effectiveness of the therapy process.

In one embodiment, the AI algorithm employed in step (a) utilizes computer vision techniques to analyze non-verbal cues, facial expressions, or physiological signals obtained from the user device to assist in the identification of the user's psychological problem.

Another embodiment integrates computer vision techniques into the AI algorithm employed in step (a) to analyze non-verbal cues, facial expressions, and physiological signals obtained from the user device. By incorporating computer vision, the system enhances its ability to identify and understand the user's psychological problem.

Computer vision techniques are utilized to capture and analyze non-verbal cues, such as facial expressions, body language, and gestures, to gain insights into the user's emotional state and identify potential psychological issues. The system employs facial expression recognition algorithms to detect and interpret facial cues, extracting information about the user's emotions, stress levels, or engagement.

Additionally, the AI algorithm utilizes physiological signals obtained from sensors integrated into the user device to further assist in the identification of the user's psychological problem. Physiological signals, such as heart rate, electrodermal activity, or brainwave patterns, are analyzed using signal processing and pattern recognition algorithms to detect patterns or anomalies that may indicate specific psychological conditions.

By leveraging computer vision techniques, the system provides a more comprehensive assessment of the user's psychological state, enabling accurate identification of the psychological problem. This information is then utilized in subsequent steps of the therapy process to determine the target outcome and personalize the therapy media selection, ultimately improving the effectiveness and tailored nature of the therapy provided.

Another embodiment of AI enhances the therapy media selection process by incorporating collaborative filtering techniques into the AI algorithm employed. Collaborative filtering enables the system to provide personalized recommendations of therapy media based on the user's psychological state, preferences, and the preferences of similar users.

Collaborative filtering leverages the collective knowledge and behaviors of a user community to recommend therapy media that align with individual preferences. The AI algorithm analyzes user feedback, ratings, and consumption patterns to identify users with similar psychological profiles or preferences. By identifying these similarities, the system can suggest therapy media that have been positively received by similar users, increasing the likelihood of personalized and relevant recommendations.

The collaborative filtering technique can be implemented using both user-based and item-based approaches. In the user-based approach, the system identifies users with similar psychological profiles and recommends therapy media that have been positively rated or engaged with by those users. In the item-based approach, the system identifies therapy media that have similar attributes or content as those previously rated positively by the user or similar users.

By incorporating collaborative filtering into the AI algorithm, the system improves the personalization of therapy media recommendations, ensuring that users receive therapy content that resonates with their psychological state and preferences, leading to enhanced engagement and treatment outcomes.

Another AI embodiment utilizes deep learning neural networks to analyze the semantic content of media and generate similarity scores to match therapy media with the user's identified problem and target outcome. This embodiment leverages the power of deep learning neural networks in the AI algorithm employed to analyze the semantic content of therapy media. Deep learning neural networks enable the system to extract high-level semantic features from media content and generate similarity scores to match therapy media with the user's identified problem and target outcome.

The AI algorithm utilizes deep learning architectures, such as convolutional neural networks (CNNs) or recurrent neural networks (RNNs), to process and analyze the audio, textual, or visual content of therapy media. These deep learning models are trained on large volumes of labeled data to learn meaningful representations of the semantic content.

By extracting semantic features from therapy media, the system can assess the similarity between media content and the user's identified problem and target outcome. Similarity scores are computed based on the learned representations, enabling the system to rank therapy media based on their relevance to the user's specific therapy needs.

Deep learning neural networks also enable the system to capture intricate relationships and contextual information within therapy media, further enhancing the accuracy and personalized nature of the media selection process. The AI algorithm continually learns and updates its understanding of the semantic content of therapy media, ensuring that the recommendations align with the user's identified problem and target outcome.

Another AI Embodiment utilizes reinforcement learning techniques to adaptively determine the target outcome based on real-time user feedback and treatment progress, thereby improving personalized therapy delivery. This method incorporates reinforcement learning techniques into the AI algorithm employed in steps (a) and (b) to adaptively determine the target outcome based on real-time user feedback and treatment progress. Reinforcement learning enables the system to optimize the therapy delivery process and enhance its personalization capabilities.

The AI algorithm utilizes reinforcement learning models, such as deep Q-networks (DQNs) or policy gradient methods, to learn from user interactions and dynamically adjust the target outcome based on treatment progress and user feedback. The system continually evaluates the effectiveness of the selected target outcome and makes adjustments to optimize the therapy experience.

During therapy sessions, the system monitors user responses, engagement levels, and treatment outcomes. Reinforcement learning techniques enable the AI algorithm to learn from this feedback and make data-driven decisions regarding the target outcome in real-time. By adapting the target outcome in real-time, the system can provide a more personalized and tailored therapy experience, maximizing the potential for positive treatment outcomes.

The reinforcement learning process involves learning from both positive and negative feedback, allowing the system to optimize the selection of the target outcome over time. Through iterative interactions and learning, the system becomes increasingly adept at determining target outcomes that align with the user's specific therapy needs, resulting in enhanced therapy effectiveness and user satisfaction.

Another AI embodiment further comprises a sensor, or sensor array, to capture real-time physiological data of the user, and the AI algorithm employed in steps (a) and (e) incorporates physiological data analysis techniques to assess the user's emotional and cognitive state, further enhancing the selection and recommendation of therapy media. This method further incorporates a sensor array in the user device to capture real-time physiological data of the user, enabling a comprehensive assessment of the user's emotional and cognitive state. The AI algorithm employed in steps (a) and (e) utilizes physiological data analysis techniques to leverage this information, further enhancing the selection and recommendation of therapy media.

The user device is equipped with sensors that measure physiological signals such as heart rate, skin conductance, brainwave activity, or eye movement. These sensors capture real-time data that provides insights into the user's emotional and cognitive states during therapy sessions.

The AI algorithm analyzes the captured physiological data using signal processing and pattern recognition techniques to assess the user's emotional and cognitive state. For example, changes in heart rate or skin conductance can indicate increased arousal or stress levels, while brainwave patterns may reflect specific cognitive states.

By incorporating physiological data analysis techniques into the AI algorithm, the system gains a deeper understanding of the user's emotional and cognitive responses to therapy media. This information enhances the system's ability to select and recommend therapy media that are well-suited to the user's current emotional and cognitive states, optimizing the therapy experience and increasing the potential for positive treatment outcomes.

Another AI embodiment employs a hybrid recommender system combining collaborative filtering, content-based filtering, and knowledge-based techniques to deliver therapy media that considers user preferences, contextual relevance, and expert knowledge. This system integrates a hybrid recommender system into the AI algorithm employed in steps (e) and step (d) to provide therapy media recommendations that consider user preferences, contextual relevance, and expert knowledge. The hybrid recommender system combines collaborative filtering, content-based filtering, and knowledge-based techniques to optimize therapy media delivery.

Collaborative filtering techniques are utilized to leverage the collective knowledge and preferences of the user community to recommend therapy media that align with individual preferences. By analyzing user feedback, ratings, and consumption patterns, the system identifies users with similar preferences and recommends therapy media that have been positively received by those users.

Content-based filtering techniques are employed to consider the content and attributes of therapy media. The AI algorithm analyzes the semantic content, genre, mood, or other relevant features of the media to determine its suitability for specific therapy needs. By considering the content and attributes of the therapy media, the system can recommend media that aligns with the user's identified problem and target outcome.

Furthermore, the AI algorithm incorporates knowledge-based techniques to leverage expert knowledge and domain-specific information in therapy media recommendations. The system utilizes expert-curated knowledge bases or ontologies to provide additional context and ensure that therapy media align with established therapeutic principles and guidelines.

By combining collaborative filtering, content-based filtering, and knowledge-based techniques, the hybrid recommender system provides therapy media recommendations that are personalized, contextually relevant, and grounded in expert knowledge. This approach enhances the effectiveness and quality of the therapy media delivered to users, resulting in improved treatment outcomes and user satisfaction.

In another AI embodiment, the user device is further configured to provide real-time interactive feedback to the user during media engagement, utilizing AI techniques to dynamically adjust the presentation of therapy media based on the user's response and engagement levels. This AI embodiment enables real-time interactive feedback to users during therapy media engagement, enhancing the therapy experience and effectiveness. The user device is configured to provide interactive feedback, and AI techniques are employed to dynamically adjust the presentation of therapy media based on the user's response and engagement levels.

During therapy sessions, the user device captures user responses, such as ratings, comments, or other forms of feedback, regarding the therapy media being presented. The AI algorithms analyze this feedback in real-time to assess the user's level of engagement, emotional response, or preference.

Based on the analysis of user feedback, the AI algorithms dynamically adjust the presentation of therapy media to optimize user engagement and treatment outcomes. For example, if the user demonstrates decreased engagement or negative emotions, the system may automatically adjust the media selection, pacing, or content to regain user interest and maintain therapeutic efficacy. Similarly, if the user responds positively or demonstrates high engagement, the system may adaptively provide additional related media or progress to more challenging therapy content.

By utilizing AI techniques to provide real-time interactive feedback and dynamically adjusting the therapy media presentation, the system personalizes the therapy experience, ensuring that users receive therapy content that is engaging, tailored to their responses, and maximizes the potential for positive treatment outcomes.

Another AI embodiment incorporates generative models, such as generative adversarial networks (GANs) or variational autoencoders (VAEs), to generate novel therapy media tailored to the user's specific psychological needs and target outcome. This AI embodiment leverages generative models, such as generative adversarial networks (GANs) or variational autoencoders (VAEs), in the AI algorithm employed in step (e) and step (d). These generative models enable the system to create novel therapy media that is tailored to the user's specific psychological needs and target outcome.

The AI algorithm employs generative models that are trained on a large corpus of therapy media to learn the underlying patterns and structures of the content. Once trained, the generative models can generate new therapy media that share similar characteristics and qualities with the existing therapy media.

By leveraging generative models, the system can create therapy media that is personalized and tailored to the user's specific therapy requirements. The generative models consider the user's identified problem, target outcome, and other contextual information to generate therapy media that address the user's psychological needs and promote the achievement of the target outcome.

The generated therapy media may include songs with lyrics, videos, or other media forms, providing novel and engaging content that is uniquely designed to resonate with the user's identified problem and target outcome.

By incorporating generative models into the AI algorithm, the system enhances its ability to deliver therapy media that is specifically created for the user, maximizing the therapeutic impact and facilitating positive treatment outcomes.

Preferably, these AI embodiments are performed under the guidance of a licensed therapist or trained medical professional. A licensed therapist or trained medical professional can spot issues with AI and make sure the entire process is successful as possible for the user or patient. While AI can operate completely independently and autonomously, it is envisioned the best use of AI is as a tool for the therapist or medical professional in helping the user or patient.

### Example

Below is an example of how the process works using a real patient without disclosing the patient's identifying information. To show multiple features of the invention, the client “Simeon” is actually an amalgamation of several patients. In this example, a client, or user logs onto the network or visits a psychologist's office. The client presents with depression, an addiction to pornography, overuse of alcohol, and occasional use of other substances or illegal drugs. He has been in multiple relationships in the past 5 years but cannot seem to commit to the woman he is currently dating. He had verbally abusive, sometimes physically abusive, and often both physically and emotionally absent, but a high-achieving father. His mother was described as overwhelmed, and caring, but often “checked out” emotionally. The patient did not internalize loving, caring, affectionate, self-disciplined, self-regulated, present parents. In his mind, what the client needs most, is a loving, caring, committed, present, spiritual, aware girlfriend/wife. The girlfriend or wife ought to value him as a person, and as a man, not as a commodity to keep buying her gifts or stay inebriated with her in order to have a “functional” (not functional) relationship. He experienced trauma from his childhood to the point that he does not have much trust in the consistent, continued goodness of others or of himself.

While the client desires a true spiritual and high-functioning psychologically conscious life, he is currently operating mostly from his egotism. Egotism is a state where the underlying emotion is fear, of not being enough, of feeling unworthy, not having enough, and a fear of “death of the ego.” When you grow up with an extreme or moderate sense of “lack” then your mind can get childhood post-traumatic stress disorder (CPTSD). It is difficult to self-regulate and to try to overcompensate for the lack, the client has done all that he could to try to achieve more, earn more, try to feel superior to others, is always looking for an angle to try to get something he perceives that he “needs” from situations or people. There is always this sense of “not enough yet.”

In the past, the client would use pornography or alcohol or indulge in depressed states of being in order to try to get a quick “high.” Like the child born to a mother on crack, the body craves that substance at birth until it is weaned off and helped, the client, like many others can be chemically addicted to this low-grade “depression” because it gives one this nostalgic high, chemically, of returning to the familiar state of “home” experience in childhood. This is why the client can still get a “high” chemically in his brain, from indulging in a “victim mode,” even though this mode leaves him more powerless to change.

The method is designed to provide the client with a new emotional home or thought pattern. The problem to overcome is the client is addicted to the dopamine highs of pornography, social media, work achievements, daredevil hobbies, promiscuity, and overuse of alcohol and substances. Alcohol is a depressant. Pornography, as science is now finding out through brain spectral imaging, burns out the pleasure center of your brain and adds to erectile dysfunction, and a general non-ability to derive pleasure from normally enjoyable and pleasurable things. These normally enjoyable and pleasurable things include nature, walks, conversations, friendships, and quietly enjoying one's own company. The method determines the source of the issue and seeks ways to address it by adopting a new more empowering thought pattern.

The client is highly creative and has adapted so far in life by acting upon many of the internalized positive traits of his parents as well. He knows how to sustain a good living, in spite of his other problems. He is tired of these maladaptive patterns in his life, this is why he took the positive action of seeking therapy, but because his parents did not model to him something more conscious than looking for constant but illusory ego gratification, he had not yet found a lasting solution! In addition to this, because he is constantly living to try to keep filling up an incessantly empty and demanding ego, he attracts, over and over, the same type of woman who mostly wants to use and manipulate him for her own egotism goals. “Like attracts like.” Until Simeon shifts his core thought pattern of operating from egotism to “Awareness”, he will keep getting the same results as before but in varying forms/disguises.

First, the method identified the problem(s) of a patient, Simeon. Second, the method identified a target outcome the patient needs to overcome the problem(s). The third step is to determine at least one thought the patient needs to overcome the problem. Specifically, the thoughts that this patient, needs to overcome is that things will never change. The thought that he is a perpetual victim, as he has actually been creating and recreating this experience since he internalized it in childhood. The never satisfied egotism “needs” and the ego needs of others that he keeps alternatively rejecting, abandoning, or trying to fulfill and overcompensate for. His hopelessness and depression led him to go to pornography and excessive use of alcohol to try to numb out egotism's constant sense of insufficiency and demand for specialness. The “highs” were temporary, and though they made him feel something “good” in the moment, they left him more and more incompetent to spend his time and energy practicing good, loving, self and other-respecting behaviors, loving responses, etc.

The fourth step is having the patient concentrate on the at least one thought (or thought pattern) needed to achieve the target outcome of the patient. The thought pattern described herein is a shifting of one's identity, as one of “an object” (for egotism's use, rejection, and aggrandizement) to a higher or more enlightened thought pattern. The client needed his parents to be present within themselves as aware, conscious, caring human beings. They had their problems from early childhood and from not internalizing healthy models of consciousness and insight for this, or even knowing they could live a higher state of thought or existence. The thought pattern change must be one that moves us out of a victim position. The method using AI, or treating professionals, identifies where the patterns derived from, and the thought pattern needed to solve the problem. The solution thought pattern is to forgive and not stay in a state of blame and victimhood towards and in relation to parents or guardians, and to move out of unconscious reactions of egotism to awareness and deeper states of presence, self-responsibility, and higher consciousness.

Step five is finding at least one song with lyrics that correspond to the thinking needed to achieve the target outcome of the patient. The songs can be chosen by the system using AI to search for lyrics that best correspond to the patient's new thought pattern or chosen based on the intuition of the therapist. In this example, the therapist used her intuition and choose Queen's “Play the Game of Love” and Coldplay's song “The Scientist.” Fredy Mercury, the singer of Queen and author of most of their lyrics, was an avid studier of esoteric philosophy, psychology, and spiritual teachings. Many musicians are skilled at this, sometimes without realizing that this is a developed skill they have!

Eckhart Tolle explains this phenomenon so excellently in his book “The Power of Now”:
“The mind is essentially a survival machine. Attack and defense against other minds, gathering, storing, and analyzing information—this is what it is good at, but it is not at all creative. All true artists, whether they know it or not, create from a place of no-mind, from inner stillness. The mind then gives form to the creative impulse or insight. Even the great scientists have reported that their creative breakthroughs came at a time of mental quietude.
The surprising result of a nation-wide inquiry among America's most eminent mathematicians, including Einstein, to find out their working methods, was that thinking “plays only a subordinate part in the brief, decisive phase of the creative act itself.” So I would say that the simple reason why the majority of scientists are not creative is not because they don't know how to think but because they don't know how to stop thinking!”

The music lyrics can be chosen to address the need for mental quietude and help the patient stop thinking and effortlessly focus on a more empowering thought pattern. This is why the disclosed methodology of accessing, cataloging, and matching these songs with a patient's problems can be so effective in treating psychological conditions. Einstein says you cannot solve a problem with the same thought pattern that created the problem. These methods are accessing a higher form of consciousness conveyed in art, poetry, verse, and song, and matching it with the patient so that they can get the psychological help they are looking for.

The sixth step is having user anchor the at least one song with the patient achieving the target outcome. In this example, this step was performed with the help of a trained professional therapist. The client needs to shift from a thought pattern of objectified and objectifying egotism to a thought pattern of innate value and worth. The method or therapist teaches the client psychological truths through the vehicle of songs. The client then listens to the song later (after our “song didactic teaching contemplation/meditation.”) Throughout the Bible there are verses saying to “meditate on these truths and principles day and night, teach them to your kids as they wake up and as they go to sleep”—these are the two states when our subconsciousness is most porous and open to accept new thought patterns.

Egotism is made up of overactive habitual, repetitive narratives from childhood. The mind/egotism is basically using up our conscious being-ness to keep feeding its own prerogative. This lyric of Freddy Mercury is inviting the client to shift into a more detached state from the narcissistic egotism thought patterns and shift into a thought pattern of “heart” or “soul”—hence why we have a whole “soul music” genre.

This leads us to the final step which is treating the patient by having the patient play the song multiple times, or at least more than two times to achieve the targeted outcome.

Now that the teaching is anchored in, the client hears these two songs in a completely new way! The client finally has a clear direction and clear heart and mind, to see who his real negative thought “opponents” are—and gracefully engage in walking the other direction into a more positive thought pattern.

The client writes his own insights from the song in his journal or on the computer application and meditates on the changes he himself wants to take part in activating. The client is motivated by a higher thought pattern of “Love” instead of the previous thought patterns of constant fear, comparing, and “I can't cope” of egotism. That is just the wounded child who wasn't taught how to cope but to constantly take shortcuts. The child didn't learn that he or she was worth more than the shortcut. The method utilizes the soothing tone and lyrics of the music to nurse the user back to health by achieving a more empowering thought problem to eliminate psychological issues!

With the computer app, the user or clinicians can type in the keywords of our clients and find a list of songs and lyrics that will best match the problem and new pattern solution required specifically for the client or use. The app can utilize Artificial intelligence as a tool. The AI provides a database that has poems, verses, and lyrics that will be perfect for helping each of the clients. The AI can take user feedback to make sure the songs are soothing, powerful, or nonabrasive while effective in treatment. The machine learning can then check the historical efficacy of each song and improve the treatment by choosing songs that have a proven track record of achieving the new thought pattern needed for improvement.

AI can be utilized as a tool, for accessing art “media” used for healing. The client can then use the app at home to write down their progress, and keep track of time dedicated to just sitting and meditating on these new thought patterns and self-concepts. They can write what surfaces for them and bring it back to therapy so we keep staying on this one song multiple times, say at least two or more times, and then later determine if there is another song, like a progression, that can help the client move upwards and onwards on his treatment. The goal is to measure the treatment efficacy of the music and then decide if the music is helping. If there is a consistent improvement, the user or client should continue with the song, if not, the client ought to pick another song. The AI can quickly access and search the database of past successes along with the client's current and desired thought patterns to decide when a new song is best suited for the user's needs.

What is claimed is:

1. A method to treat a psychological issue of a user by providing customized therapy media to the user based on the psychological state of the user, the method comprising:
a) defining a problem of a user;
b) determining a target outcome, the user needs to overcome the problem;
c) selecting at least one thought the user needs to overcome the problem;
d) having the user concentrate on the at least one thought the user needs to overcome the problem and achieve the target outcome of the user;
e) searching at least one media that corresponds to the thinking needed to achieve the target outcome of the user, wherein the media is selected from a song with lyrics and a video;
f) having the user anchor to the at least one media with the user achieving the target outcome; and
g) treating the user by having the user playing the media multiple times to achieve the targeted outcome.

2. The method of claim 1, further comprising creating a video of the selected at least one media where the at least one media is a song with lyrics and treating the patient by having the patient play the video multiple times to achieve the targeted outcome.

3. The method of claim 1, further comprising determining effectiveness of the at least one media in achieving the target outcome and changing the at least one media.

4. The method of claim 1, further comprising determining effectiveness the at least one thought the user needs to overcome the problem and changing the at least one thought the user needs to overcome the problem.

5. A computer-implemented method of providing a customized therapy media to a user based on psychological state of the user, the method comprising:
a) identifying, via a user device connected to a server over a network, a psychological problem of the user;
b) determining, from a database connected to the server, a target outcome for the user to overcome the problem as identified;
c) selecting, by the server, at least one thought corresponding to the target outcome the user needs to overcome the problem;
d) searching, in a media database coupled to the network and having a plurality of media, one or more media that correspond to the problem and the target outcome the user needs using artificial intelligence; and
e) anchoring the user, via the user device, to at least one media from said one or more media to achieve the target outcome.

6. The computer-implemented method as claimed in claim 5, wherein the step of determining at least one thought includes determining at least one alternative thought the user needs to achieve the target outcome.

7. The computer-implemented method as claimed in claim 5, wherein the step of anchoring includes playing the at least one media multiple times in loop till the target outcome is achieved.

8. The computer-implemented method as claimed in claim 5, wherein the one or more media includes a plurality of audio/video material including audio/video teaching songs with lyrics.

9. The computer-implemented method as claimed in claim 5, further comprising receiving from the user device, a compliance report on listening to the at least one media by automatically acquiring a daily report.

10. The computer-implemented method as claimed in claim 5, wherein the step of identifying the problem includes:
receiving from the user, via the user device, responses to a psychological evaluation test provided to the user on the user device, wherein the user device is adapted to receive user inputs via a graphical user interface.

11. The computer-implemented method as claimed in claim 5, wherein the step of determining the target outcome comprises comparing, with a database of known psychological conditions and their respective traits, the identified problem of the user.

12. The computer-implemented method as claimed in claim 5, wherein the step of anchoring the user comprises setting a duration for which the at least one media is to be played to achieve the target outcome.

13. The computer-implemented method as claimed in claim 5, further comprising receiving a review response from the user via the user device on the at least one media to improve the step of searching of relevant media through machine learning.

14. The computer-implemented method as claimed in claim 13, further comprising monitoring a variation in the psychological state of the user at regular intervals in course to achieve the target outcome.

15. The computer-implemented method as claimed in claim 5, further comprising receiving a response from the user while focusing on the at least one thought or an alternative thought; and changing the media based on a change in the at least one thought or the alternative thought.

16. The computer-implemented method of claim 5, further comprising using artificial intelligence by the server over the network to identify the psychological problem of the user.

17. The computer-implemented method of claim 5, further comprising using artificial intelligence by the server to determine from the database the target outcome for the user to overcome the problem as identified.

18. The computer-implemented method of claim 5, further comprising using artificial intelligence by the server to select the at least one thought corresponding to the target outcome the user needs to overcome the problem.

19. The computer-implemented method of claim 5, further comprising using artificial intelligence by the server to search and to select the at least one media from said one or more media in a media database to achieve the target outcome.

20. A system for providing a customized therapy media to a user based on psychological state of the user, said system comprising:
a non-transitory storage medium; and
a processor coupled to the non-transitory storage medium, configured at least to:
identify, via the user device, a psychological problem of the user;
determine, from a database connected to the server, a target outcome for the user to overcome the problem as identified;
select, by the server, at least one thought corresponding to the target outcome the user needs to overcome the problem;
search, in a media database coupled to the network and having a plurality of media, one or more media that correspond to the problem and the target outcome the user needs using artificial intelligence; and
anchor the user, via the user device, to at least one media from said one or more media to achieve the target outcome.
